(19) United States
(12) Patent Application Publication
(10) Pub. No.: US 2026/0157749 A1
(43) Pub. Date: Jun. 11, 2026

(54) SYSTEMS AND METHODS FOR ENDOSCOPIC TISSUE APPOSITION AND SUTURING

(71) Applicant: INTUITIVE SURGICAL OPERATIONS, INC., Sunnyvale, CA (US)

(72) Inventor: Matthew D. Rohr Daniel, San Francisco, CA (US)

(21) Appl. No.: 19/125,669

(22) PCT Filed: Oct. 27, 2023
(86) PCT No.: PCT/US2023/078007

§ 371 Date: Apr. 29, 2025

Related U.S. Application Data
(60) Provisional application No. 63/421,029, filed on Oct. 31, 2022.

(51) Int. Cl. A61B 17/04

(52) U.S. Cl. CPC A61B 17/0469

(57) ABSTRACT

An apparatus comprises a housing including an instrument interface. The instrument interface is configured to releasably engage a distal portion of an endoscope. The apparatus also comprises a drive system coupled to the housing and configured to releasably engage a drive element extending from the endoscope. The apparatus may also comprise an arc-shaped needle coupled to the drive system and a tissue apposition system. The tissue apposition system may comprise a tissue chamber in the housing across which a trajectory of the arc-shaped needle extends and a tissue biasing component configured to draw tissue into the tissue chamber.

### CROSS-REFERENCED APPLICATIONS

This application claims priority to and benefit of U.S. Provisional Application No. 63/421,029 filed Oct. 31, 2022 and entitled “Systems and Methods for Endoscopic Tissue Apposition and Suturing,” which is incorporated by reference herein in its entirety.

### FIELD

Examples described herein relate to systems and methods for endoscopic tissue apposition and suturing. More particularly, examples may relate to tissue apposition and suturing during an endoscopic procedure.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. Some minimally invasive medical instruments may be used to perform endoscopic tissue apposition and suturing. Systems and methods are needed to provide effective tissue apposition and suturing.

### SUMMARY

The following presents a simplified summary of various examples described herein and is not intended to identify key or critical elements or to delineate the scope of the claims.

In some examples, an apparatus may comprise a housing including an instrument interface. The instrument interface may be configured to releasably engage a distal portion of a flexible elongate body, such as an endoscope. The apparatus may also comprise a drive system coupled to the housing and configured to releasably engage a drive element extending from the endoscope. The apparatus may also comprise an arc-shaped needle coupled to the drive system and a tissue apposition system. The tissue apposition system may comprise a tissue chamber in the housing across which a trajectory of the arc-shaped needle extends and a tissue biasing component configured to draw tissue into the tissue chamber.

In some examples, an instrument system may comprise a flexible instrument or device body comprising a distal end portion. The distal end portion may comprise a housing, a gear system within the housing, an arc-shaped needle coupled to the gear system for bi-directional motion of the arc-shaped needle relative to the housing, and a tissue apposition system. The tissue apposition system may comprise a tissue chamber in the housing across which a trajectory of the arc-shaped needle extends and a tissue biasing component configured to draw tissue into the tissue chamber.

In some examples, an instrument system may comprise a flexible instrument or device body including a plurality of working channels configured to receive one or more instruments and a drive element extending in a first one of the plurality of working channels. The instrument system may also comprise a housing configured to engage a distal portion of the flexible device body and a drive system extending within the housing. The drive system may be configured to couple to a distal end of the drive element. The instrument system may also comprise an arc-shaped needle coupled to the drive system. The arc-shaped needle may be configured for bi-directional motion. The instrument system may also include a tissue apposition system including a tissue chamber in the housing across which a trajectory of the arc-shaped needle extends and a tissue biasing component configured to draw tissue into the tissue chamber.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1A illustrates a side view of a distal portion of an instrument system with a suture apparatus, according to some examples.

FIG. 1B illustrates a side view of a distal portion of an instrument system with a suture apparatus, according to some examples.

FIG. 2 illustrates an exploded view of a distal portion of an instrument system with a suture apparatus, according to some examples.

FIG. 3 illustrates an end view of a distal portion of the suture apparatus of FIG. 2, according to some examples.

FIG. 4 illustrates a side view of a distal portion of the instrument system of FIG. 2 with the suture apparatus, according to some examples.

FIG. 5 illustrates a gear system of the suture apparatus of FIG. 2, according to some examples.

FIGS. 6A and 6B illustrate a needle system for use with a suture apparatus, according to some examples.

FIG. 7A illustrates tissue engagement features at a distal end of a suture apparatus, according to some examples.

FIG. 7B illustrates a side view of tissue in apposition with a needle and tissue engagement features, according to some examples.

FIG. 7C illustrates tissue engagement features at a distal end of a suture apparatus, according to some examples.

FIG. 7D illustrates a side view of a distal portion of an instrument system with a suture apparatus, according to some examples.

FIGS. 7E-7G illustrate a suture apparatus, according to some examples.

FIG. 8 illustrates a gear system of a suture apparatus, according to some examples.

FIG. 9 illustrates a side view of a distal portion of an instrument system with a suture apparatus, according to some examples.

FIG. 10 is a flowchart illustrating a suturing procedure.

FIG. 11 is a simplified diagram of a patient anatomy, according to some examples.

FIG. 12 is a robotically-assisted medical system, according to some examples.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

The technology described herein provides techniques and treatment systems for tissue grasping, apposition, and suturing. Although the examples provided herein may be used for suturing of stomach tissue as in endoscopic sleeve gastroplasty (“ESG”), it is understood that the described technology may be used in performing procedures in artificially created lumens or any endoluminal passageway or cavity, including in a patient trachea, colon, intestines, stomach, liver, kidneys and kidney calices, brain, heart, circulatory system including vasculature, fistulas, and/or the like. In various examples, flexible instrument systems may include a suture apparatus that may fold, bend, pinch, or otherwise cause apposition of tissue such that two portions of tissue are brought close to each other or into contact with each other in preparation for delivering a suture through the tissue.

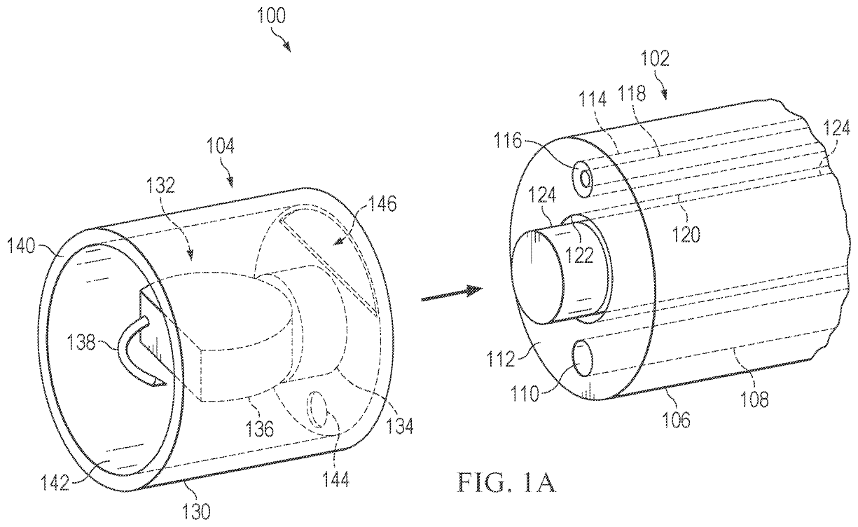

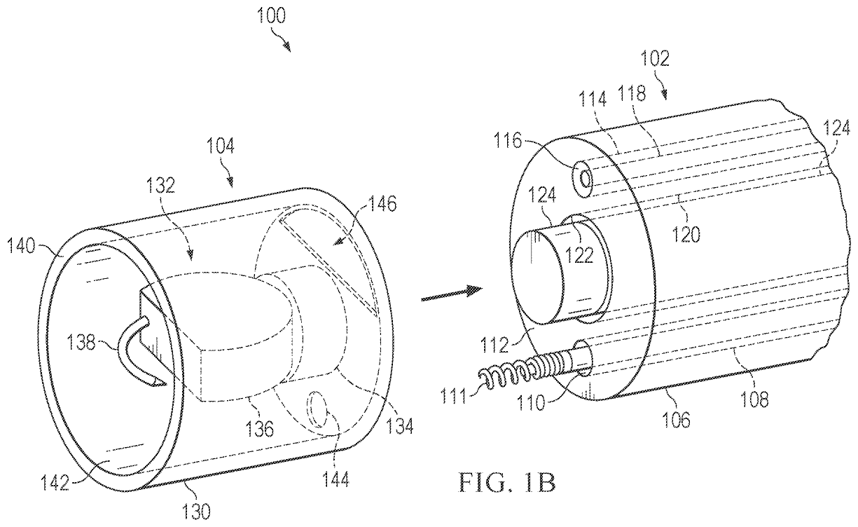

FIG. 1A illustrates a side view of a distal portion of an instrument system 100 including an elongate flexible device 102 and a suture apparatus 104. In some examples, the instrument system 100 may be an endoscopic instrument system and the elongate flexible device 102 may be a steerable endoscope, gastroscope, etc. The device 102 may serve as a platform to drive motion of mechanisms in the suturing apparatus, introduce working components into the anatomic structures accessed by the instrument system 100, and capture image data of the anatomic structures. The device 102 may include a flexible body 106 through which extends one or more working channels 108. The working channels 108 may extend through the flexible body 106 to provide passage for removable instrument systems and allow instruments to be exchanged during a procedure. The working channels 108 may also or alternatively allow fluid passage, deliver vacuum pressure, or otherwise provide access between proximal and distal portions of the elongate flexible device 102. Each working channel 108 may define an opening 110 in a distal end portion 112 of the device 102.

An imaging channel 114 may also extend through the flexible body 106 and terminate at an opening 116 to provide passage for an imaging system 118 (e.g., imaging system 909 or a component thereof). The imaging system 118 may be an integrated component of the device 102 (e.g., permanently coupled) or may be slidably received within and removable from the channel 114. In some examples where the imaging system 118 is permanently coupled, the imaging system 118 may be articulatable relative to the device 102, while in other examples, the imaging system 118 may be fixed relative to the device 102 when permanently coupled. In some examples, the imaging system 118 may transmit images using one or more flexible optical fibers. Digital image-based imaging systems may have a “chip on the tip” design in which a distal digital sensor such as a one or more charge-coupled device (CCD) or a complementary metal oxide semiconductor (CMOS) device store image data. The imaging system 118 may capture two- or three-dimensional image data. For example, stereo imaging systems may employ stereo cameras to capture stereo images of the patient anatomy. In some examples, the imaging channel may be omitted, and an imaging system may be delivered through the working channel 108.

A drive channel 120 may also extend through the flexible body 106 and terminate at an opening 122. The drive channel 120 may provide passage for a drive element 124. The drive element 124 may be an integrated component of the device 102 (e.g., permanently coupled) or may be slidably received within and removable from the channel 120. In some examples where the drive element 124 is permanently coupled, the drive element 124 may be axially and rotationally articulatable relative to the device 102, while in other examples, the drive element 124 may be fixed axially relative to the device 102 while rotationally articulatable. In some examples, the drive element 124 may include a flexible torque drive shaft that allows for bending with the flexible body 106 while delivering torque from a motor or other drive system coupled to a proximal end of the drive element. In some examples, the drive channel may be omitted, and the drive element 124 may be delivered through a working channel 108. In some examples, the drive element 124 may be coupled to a motor of a robot-assisted manipulator (e.g., manipulator assembly 902).

In some examples, the suture apparatus 104 may be permanently coupled to or integrally formed with the device 102. In other examples, the suture apparatus 104 may be removably coupled to the device 102. For example, the device 102 may be a flexible, steerable endoscope, colonoscope, duodenoscope, gastroscope, or the like (which will collectively be referred to as an endoscope herein), and the suture apparatus 104 may be a removable attachment that removably couples to a distal end of the device 102. The suture apparatus 104 may include a housing 130 that supports and provides cover for at least a portion of a needle drive system 132. The needle drive system 132 may include a drive input 134 that couples to the drive element 124 of the device 102 to transmit a torque from the drive element 124. The needle drive system 132 may also include a gear system 136 for driving motion of a needle 138. The gear system 136 may be coupled between the needle 138 and the drive input 134. The gear system 136 may include a gear train including one or more gears to transmit torque from the drive input 134 to drive motion of the needle 138. The needle 138 may have an arc or arcuate shape and may rotate to sweep through apposed tissue positioned within or adjacent the housing 130. The housing 130 may include a distal engagement surface 140 that may contact, seal with, or otherwise engage a tissue surface during a suturing procedure. The housing 130 may also include a tissue apposition system including a tissue chamber 142 and a tissue biasing component 144. The tissue chamber 142 defines a space into which tissue may be drawn in a folded, bended, pinched, or otherwise apposite configuration. The tissue biasing component 144 may be, for example, one or more vacuum sources or vacuum ports that transmit a biasing force to draw tissue into the tissue chamber 142. For example, the tissue biasing component 144 may be a port with an aperture that couples to the working channel through which a vacuum may be applied to the suture apparatus 104. In some examples, the tissue biasing component may include multiple redundant vacuum ports and channels to maintain vacuum pressure if one port or channel becomes clogged. In various embodiments, the multiple redundant vacuum ports may interface with a single working channel 108 or the multiple redundant vacuum ports may interface with separate working channels 108.

In some examples, as shown in FIG. 1B, the tissue biasing component of the tissue apposition system may include a tissue apposition tool 111 to aid with drawing tissue into the tissue chamber 142. The tissue apposition tool 111 extends through a working channel in the device 102 and through the housing 130 to engage target tissue. For example, the tissue apposition tool 111 may extend through working channel 108 in the device 102 and extend through a port or aperture in the housing 130 that couples with the working channel 108, with the tissue apposition tool 111 further extending distally of the housing 130 to contact target tissue. In some embodiments, the tissue apposition tool 111 may extend through the aperture forming the tissue biasing component 144 while in other embodiments the tissue apposition tool 111 may extend through a separate aperture in the housing 130. In some examples, a tissue apposition tool 111 includes a tissue engaging component in the form of a helical shaped component that extends from the working channel 108 of the device 102, through the housing 130, and towards a tissue surface. The helical shaped component may be pushed forward against the tissue while being rotated such that the helical shaped component drives into the tissue in a corkscrew movement. Once the helical shaped component has been driven into the tissue to a desired depth, the helical shaped component can be pulled back without twisting, bringing the tissue into the tissue chamber 142 and into the suturing needle path. In some examples, the tissue apposition tool 111 may include a tissue engaging component with an additional or alternative configuration than a helical shaped component, such as opposed jaws, clamps, or other configurations. In some examples, multiple tissue apposition tools 111 may be used, and the multiple tools may have a helical shaped component, opposed jaws, clamps, or other configurations. In some examples, the tissue apposition tool 111 may be used as part of the tissue biasing component in addition to vacuum apposition as described herein to draw tissue into the tissue chamber 142. In other examples, the tissue apposition tool 111 may be used to draw in tissue without vacuum apposition. In yet other examples, vacuum apposition may be used without the tissue apposition tool 111 and the tissue apposition tool 111 may be omitted.

The housing 130 may also provide a viewing port 146 which may include a channel or open space through the housing 130 that allows the imaging system 118 to capture images of a field of view distal of the housing 130. In some examples, the viewing port may accommodate passage of imaging instruments such as portions of the imaging system 118 that may be positioned to extend distally of the flexible body 106 and into the housing 130. In some examples, portions of the imaging system 118 may be positioned to extend distally of the flexible body 106 and through and distally of the housing 130.

In some examples, a suturing procedure may be performed with the system 100. The suturing procedure may achieve a full thickness suture throw through apposed tissue including all layers of the tissue. For example, in a stomach suturing procedure, the needle and suture may penetrate all four layers of the tissue including the submucosal, mucosal, muscle layers, and serosa. In some examples, the thickness of the tissue, including the four layers, may be between approximately 2 mm and 10 mm. In some examples, the tissue thickness may be approximately 5 mm. In some examples of a suturing procedure, the distal engagement surface 140 may be placed near or in contact with a tissue surface. A vacuum may be applied through the tissue biasing component 144 to create suction that draws the tissue into the chamber 142. Additionally or alternatively to vacuum apposition, a tissue apposition tool 111 may be advanced into contact with the tissue and retracted to draw the tissue into the chamber 142. The tissue apposition tool 111 may be applied through the tissue biasing component 144 or through a separate port. With the tissue in the chamber 142, the drive element 124 may be actuated to activate the needle drive system 132 to move the arcuate needle 138 through the tissue to create a suture. The needle drive system 132 may be positioned to radially fit within an outer diameter of the housing 130 while portions of the needle drive system 132 may extend beyond an axial length of the housing 130. In addition, the trajectory of the arcuate needle 138 may be such that the needle 138 radially fits within the outer diameter of the housing 130 while portions of the needle 138 may extend beyond the axial length of the housing 130 in use.

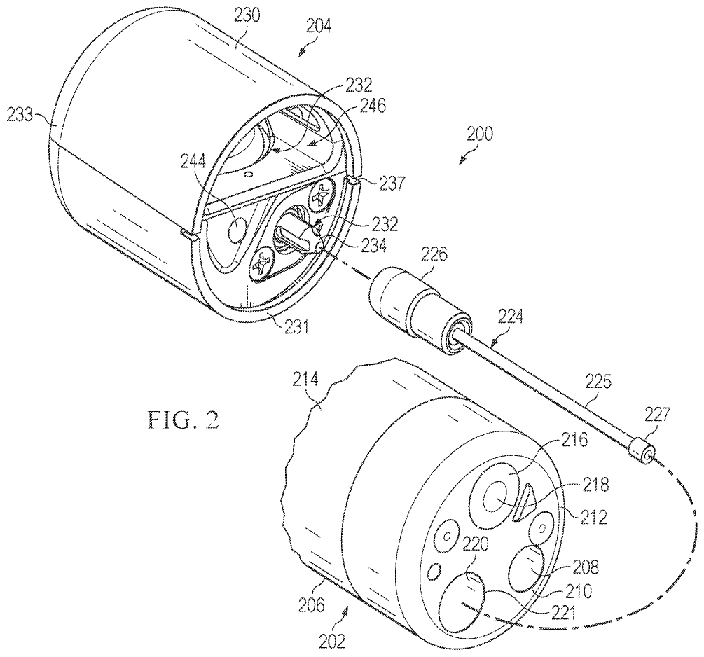

FIG. 2 illustrates an exploded view of a distal portion of an instrument system 200 including an elongate flexible device or instrument 202 (e.g., an example of an elongate flexible device 102) and a suture apparatus 204 (e.g., an example of a suture apparatus 104). In this example, the instrument system 200 may be an endoscopic instrument system, and the elongate flexible device 202 may be a steerable monoscopic endoscope (but in other examples a stereoscopic endoscope may be used). The elongate flexible device 202 may include a flexible body 206 through which extends one or more working channels 208 and one or more drive channels 220. In some examples, the elongate flexible device 202 may include one, two, three, or more working channels 208. The working channels 208 may extend through the flexible body 206 to provide passage for removable or exchangeable instrument systems such as ablation tools, biopsy instruments, imaging instruments, or irrigation tools. In some examples, one or more working channels 208 may be coupled to a vacuum source to provide suction through the working channels 208. The working channels 208 may additionally or alternatively provide passage for a tissue apposition tool 111 as described above. The working channels 208 may also or alternatively allow fluid passage or otherwise provide access between proximal and distal portions of the elongate flexible device 202. Each working channel 208 may define an opening 210 in a distal end portion 212 of the device 202. Each drive channel 220 may define an opening 221 in the distal end portion 212. In some examples, other auxiliary channels and ports may be used to provide irrigation or to introduce tools and instruments. In some examples, the elongate flexible device 202 may house cables, linkages, or other actuation controls (not shown) that extend between the proximal and distal ends of the elongate flexible device 202 to controllably bend or articulate and steer at least a portion of the elongate flexible device 202. An imaging channel 214 may also extend through the flexible body 206 and terminate at an opening 216 to provide passage for an imaging system 218 (e.g., imaging system 909 or a component thereof) which in this example may be a monoscopic imaging system.

The drive channel 220 may provide passage for a drive element 224. In this example, the drive element 224 is rotatable within and removable from the drive channel 220. In this example, the drive element 224 includes a flexible torque drive shaft 225 extending between a drive head 226 at a distal end portion of the drive element 224 and a proximal engagement portion 227 at a proximal end portion of the drive element 224. The proximal engagement portion 227 may engage an actuator (e.g., a motor) that causes rotation of the shaft 225 and drive head 226 in either or both directions about the axis of the flexible torque drive shaft 225. Additionally or alternatively, the proximal engagement portion 227 may include a handle for manual actuation by a user. The flexible torque drive shaft 225 may flexibly bend with the flexible body 206 while delivering torque to the drive head 226. In some examples, the drive element may be coupled to a gear box, at the distal end, to reduce the amount of torque required to be transmitted down the length of the flexible body 206 and to increase performance. In some examples, the drive channel 220 may be omitted, and the drive element 224 may be delivered through a working channel 208.

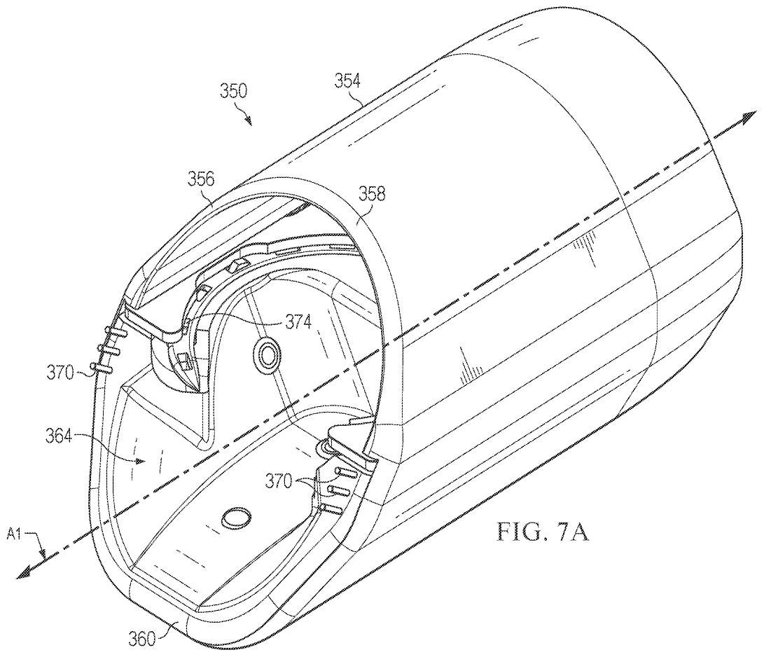

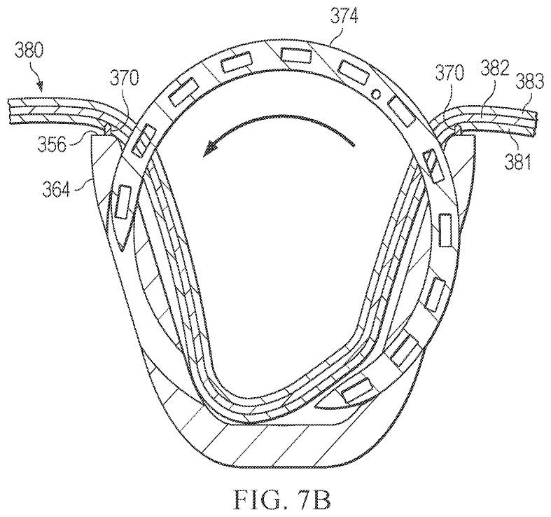

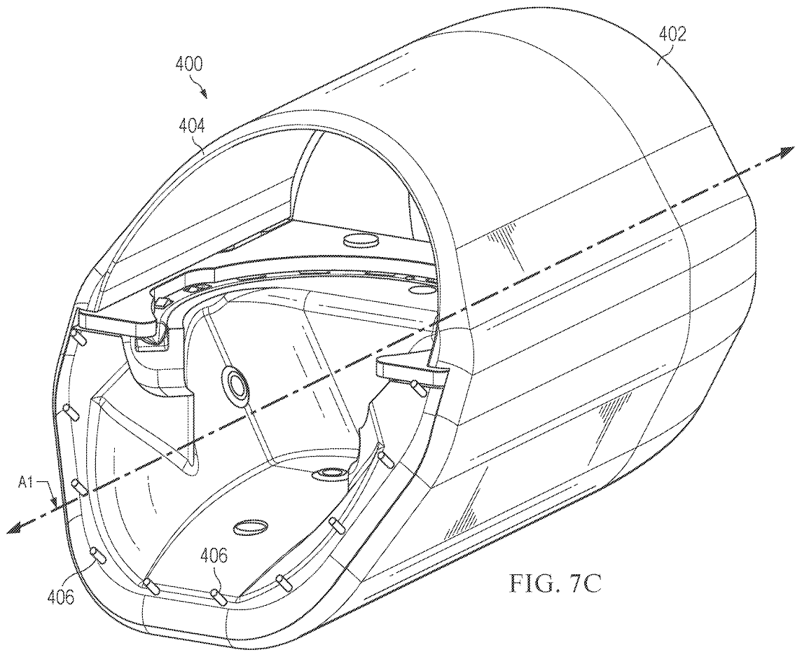

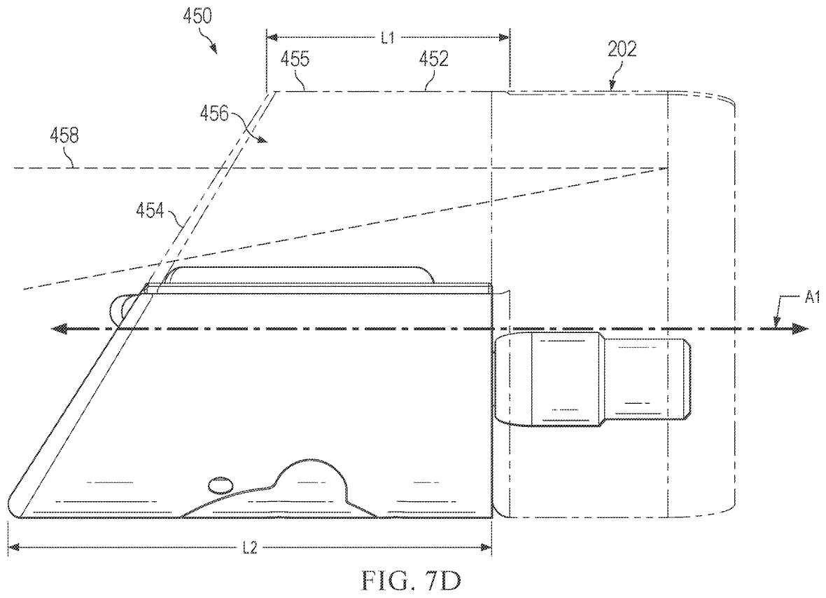

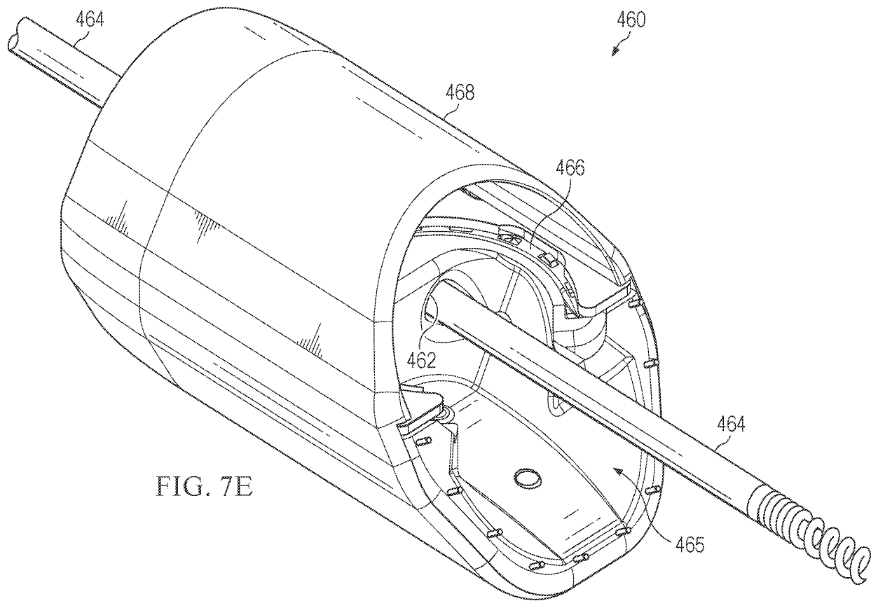

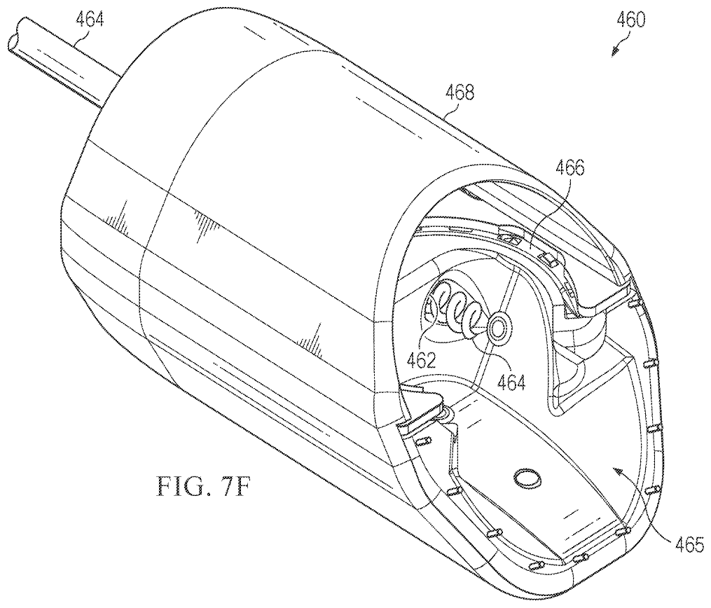

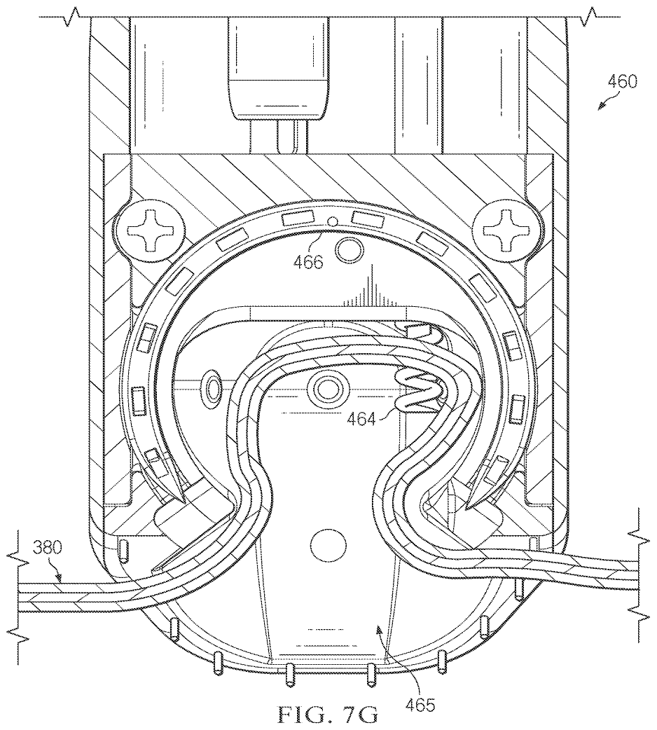

In this example, the suture apparatus 204 may include a housing 230 that supports and provides cover for at least a portion of a needle drive system 232. The needle drive system 232 may include a drive input 234 that couples to the drive head 226 of the drive element 224 and rotates in response to torque received from the drive element 224. The suture apparatus 204 may include an instrument interface portion 231 that couples to the distal end portion 212 of device 202 and a tissue interface portion 233 that engages with tissue that is distal of the suture apparatus 204. In some examples, the instrument interface portion 231 may include an attachment feature 237, such as a flange, that may press-fit to the distal end portion 212 of the device 202. The housing 230 may include a viewing port 246 which, in this example, is an arc-shaped channel through the housing 230 that allows the imaging system 218 to capture images of a field of view distal of the housing 230. The housing 230 may also include a tissue apposition system including a tissue chamber and a tissue biasing component 244. In this example, the tissue biasing component 244 may be a vacuum port that provides suction to draw tissue into a tissue chamber 242. For example, the vacuum port 244 may couple to the working channel 208 so that a vacuum applied through the working channel 208 will create suction through the housing 230 to draw tissue into the tissue chamber 242. Additionally or alternatively, a port may provide passage for a tissue apposition tool 111 to advance to engage tissue and retract to draw tissue into the tissue chamber 242 (see FIGS. 7E-7G).

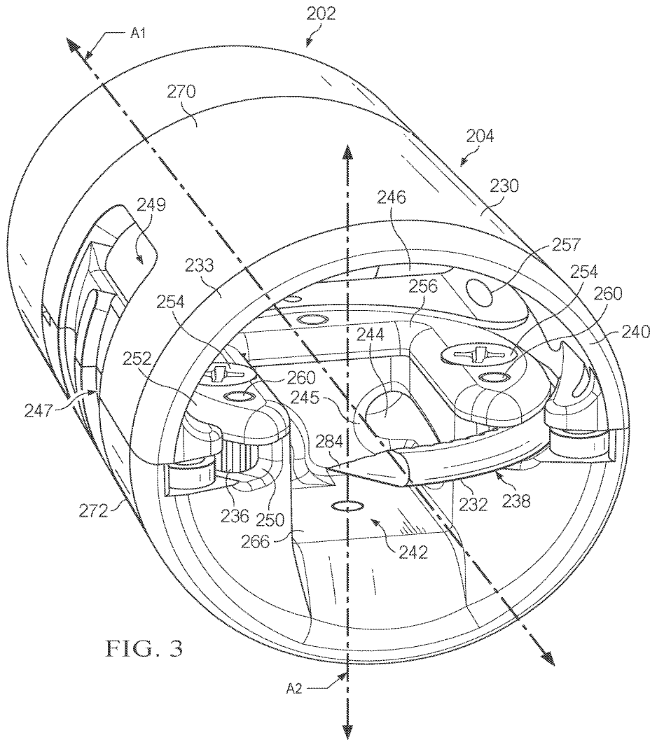

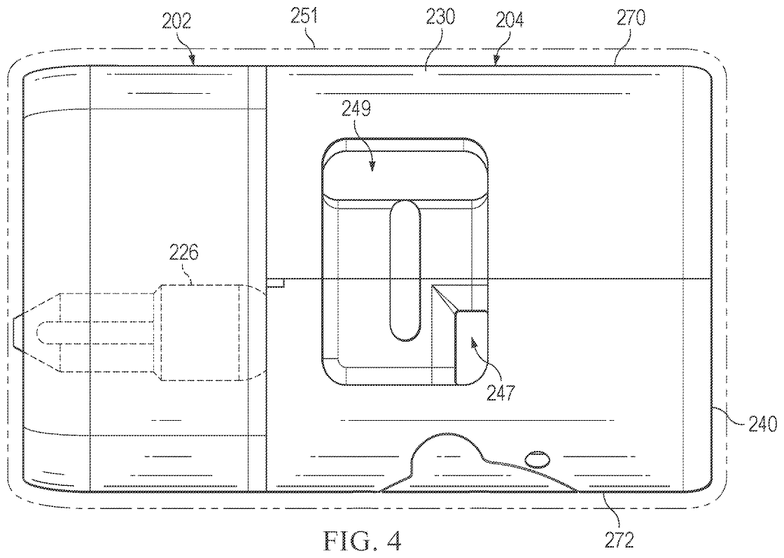

As shown in FIG. 3, the tissue interface portion 233 of the housing 230 may include an atraumatic distal engagement surface 240 that may contact a tissue surface during a suturing procedure. In this example the distal engagement surface may be generally circular and may be generally perpendicular to a central axis A1 through the housing 230. The housing 230 may also include the tissue chamber 242 into which tissue may be drawn in a folded, bended, pinched, or otherwise apposite configuration. The surfaces of the tissue chamber 242 proximal of the engagement surface 240 may be sloped, curved, or otherwise shaped to provide a smooth, atraumatic interface with the in-drawn tissue to prevent damage to the tissue. Tissue biasing port 244 may extend from the instrument interface portion 231 to an opening 245 in the tissue chamber 242 to provide vacuum pressure to the tissue chamber 242. Additionally or alternatively, a port may provide passage for a tissue apposition tool as described above. As described in further detail below, optionally, the housing 230 may include a first arcuate portion 270 and a second arcuate portion 272. As shown in FIGS. 3-4, a port 247 may extend through a side wall of the second arcuate portion 272 and a port 249 may extend through a side wall of the first arcuate portion 270. A sealing member 251, such as a heat shrink tubing may be sealed around the housing 230 such that an air flow channel or path is created from the port 249 to the port 247 and through the tissue chamber 242 to the tissue biasing port 244. This configuration of ports may bias the inward drawn tissue toward the portion of the tissue chamber 242 within the lower second arcuate portion 272. Alternatively, two or more vacuum openings (e.g., ports 244) in different areas of the tissue chamber 242 may allow for a more distributed and even application of vacuum which may allow tissue to be more evenly and deeply drawn into the chamber 242. In some examples, the housing may have a maximum outer diameter of approximately 16 mm.

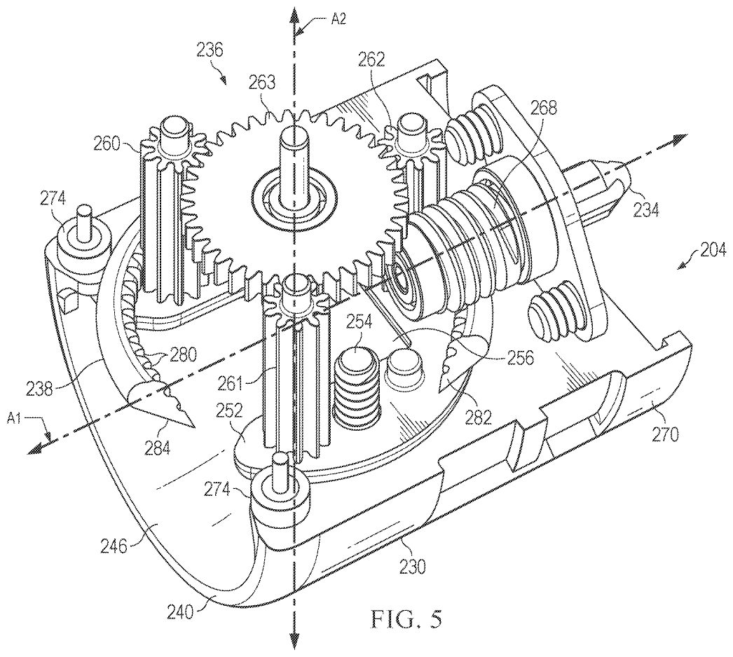

With further reference to FIG. 3 and FIG. 5, in this example, the needle drive system 232 may also include a gear system 236 for driving motion of a needle 238. The gear system 236 may be coupled between the needle 238 and the drive input 234. The gear system 236 and needle 238 may be supported within the housing 230 by a track 250. A plate 252 may be coupled to the gear system 236 to hold the gear system 236 in place on the track 250. The plate 252 may be coupled to the housing 230 by attachment members 254 such as screws. The plate 252 may be formed in a U-shape with a central gap 256 that provides space for the in-drawn tissue.

The gear system 236 may transmit torque from the drive input 234 to drive motion of a needle 238. In this example, the gear system 236 may include three drive spur gears 260, 261, 262 placed approximately equidistant from each other in a triangular configuration. A larger central spur gear 263 may engage the three drive spur gears 260, 261, 262, maintaining synchronous motion of the three gears. The triangular configuration of the three smaller spur gears is oriented such that gear 260 and gear 261 are on either side of the gap 256 and the tissue chamber 242. Gear 262 is proximal of the gap 256. When vacuum is applied and/or a tissue apposition tool retracts tissue into the tissue chamber 242, the tissue may thus be drawn into the triangular configuration, between the gears 260 and 261. The large synchronous gear 263 may rotate about an axis A2 that may be generally perpendicular to the central axis A1 of the housing 230. The gear 263 may be placed as far from the plate 252 as possible to maximize the amount of tissue drawn into the tissue chamber 242. As shown in FIG. 3, a surface 266 of the housing 230 may shield the tissue from the gear interaction points to prevent damage to in-drawn tissue. The central gear 263, for example, may be outside of the tissue chamber 242. The proximal spur gear 262 may be engaged and driven by a worm gear 268 which may be coupled to the drive input 234.

In some examples the housing may be integrally formed. In this example, to promote manufacturability, maintenance, or functionality, the housing 230 may have two coupled components, a first arcuate portion 270 and a second arcuate portion 272. Bearings 274 may provide an interface for connecting the portions 270, 272. In some examples, the bearings 274 may be omitted and replaced with a flush connection to discourage tissue ingress in the area around the gear system 236. In some examples, the first arcuate portion 270 may surround the viewing port 246 and may include a window formed of a transparent polymer or ceramic material to allow viewing of the adjacent anatomic area in the field of view of the imaging system. In some examples the entire portion 270 may be formed of the transparent material.

The needle 238 may have an arc or partially circular shape and may rotate about the axis A2 to sweep through the tissue chamber 242 and through the tissue captured within the chamber. The needle 238 may include a series of involute teeth 280 arranged around an inner circumference of the needle 238 with sizing and spacing to engage the gear system 236. In some examples, the needle 238 may extend approximately 270 degrees and engage at least two of the gears 260, 261, 262 simultaneously. The needle 238 may include a pointed end 282 and a pointed end 284 to allow the needle to be driven in either a clockwise or counter-clockwise direction. The direction of rotation of the drive element 224 may drive either the clockwise or counter-clockwise motion of the needle 238. In some examples, the trajectory of the needle may approximately bisect the tissue chamber 242. In some examples, the needle 238 may rotate in a full 360 degree circular route in a plane generally perpendicular to the axis A2 to return the suture-affixed portion of the needle 238 to a staging configuration. In some examples, the needle 238 may pivot bi-directionally in a semi-circular route (e.g., approximately 180 degrees) in a plane generally perpendicular to the axis A2 to return the suture-affixed portion of the needle to the staging configuration. In the staging configuration, the needle 238 may be rotated to a position such that the needle does not obstruct ingress of tissue to the chamber 242. For example, the pointed end 284 may be located near the gear 260 in the staging configuration. In some examples, the route of the needle 238 may extend outside of the housing 230 and distally of the distal engagement surface 240. In other examples, the route of the needle may be entirely within the confines of the housing 230. Suture material may be fixed to the needle at an attachment portion, for example, approximately equidistant from the pointed ends 282, 284. As the needle 238 is rotated about the axis A2, the needle 238 may draw the suture material through the folded tissue held within the tissue chamber 242. In some embodiments, the needle 238 may have a single pointed end (e.g., pointed end 282) and an opposite blunted end (e.g., end 284).

The housing 230 may, optionally, include one or more sensors or sensory systems 257. For example, an imaging sensor such as an ultrasound sensor, positioned on the bottom surface of the tissue chamber and pointed in the direction of A2, may be used to evaluate the shape or thickness of tissue in the tissue chamber to verify that a full thickness of tissue has been captured prior to suture. A pressure sensor may be used to confirm an expected vacuum pressure. One or more drive sensors, such as a plurality of hall effect sensors, inductive proximity sensors, and/or optical sensors functioning as an encoder may measure rotations of the needle either directly or by inference. Measurements may occur at any moving element of the drive system.

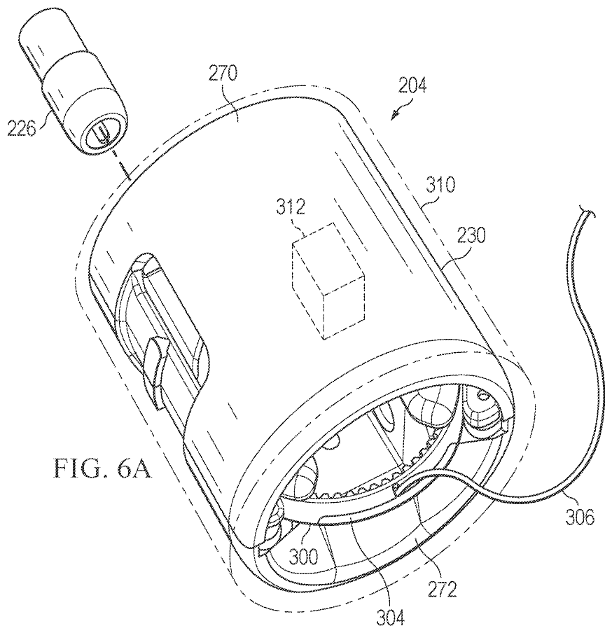

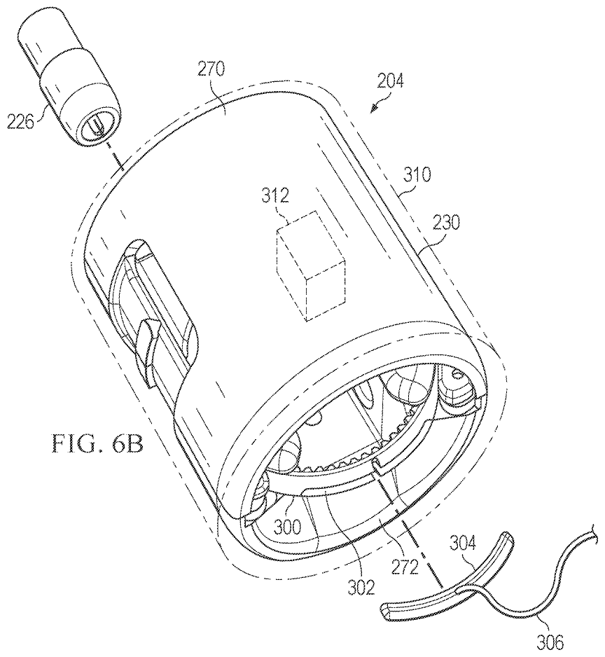

FIGS. 6A and 6B illustrate an alternative needle 300 that may be used with the suture apparatus 204. In this example, a needle 300 may be substantially similar to the needle 238 with the differences as described. The needle 300 may include a radial pocket 302 sized and shaped to receive a suture tag 304. The suture tag 304 may have an arcuate shape and may be affixed to suture material 306. When the needle 300 is driven through the tissue gathered into the tissue chamber 242, the suture tag 304 and the attached suture material 306 may pass through the tissue as a single assembly. At the conclusion of the suture pattern the suture tag 304 may be released from the needle pocket 302 such that the suture tag will act as an anchor point for the completed suture pattern. A suture tag mechanism 312 in the housing may include a suture cartridge and a suture release mechanism. In some examples, after the needle is driven in a 360 degree or 180 degree trajectory, a new suture tag moves from the suture cartridge and is deposited into the pocket 302 vacated by the previously released suture tag. In some examples, a new suture tag may be deposited in the pocket 302 by an external tool. In some examples, the suture tag may be released into the tissue by activation of the suture release mechanism. The activation may be automated to occur after the needle completes a predetermined trajectory or may be manually actuated by a clinician. In some embodiments, the suture tag 304 and the suture tag mechanism 312 may be used with the needle 504 and needle drive system 500 described in further detail below. Additionally or alternatively to the suture tag 304, another portion of the needle may be affixed to suture material and may be removable from the needle body to act as an anchor point (e.g., a distal tip of the needle or a proximal end of the needle).

In some examples, a flexible sheath or tubing 310 may extend over the suture apparatus 204 to prevent ingress or egress of fluid or tissue to the housing 230. In some examples, the tubing may be a heat shrink-fit tubing.

FIG. 7A illustrates a suture apparatus 350 according to an alternative example. In this example, the suture apparatus 350 may be substantially similar to the suture apparatus 204, with the differences as described. In this example, the suture apparatus 350 includes a housing 354 including a distal engagement surface 356 that may contact a tissue 380 (FIG. 7B) during a suturing procedure. In this example, the distal engagement surface 356 may have an irregular shape or outline, including curved portions 358 and straight portions 360. The portions 358, 360 may form an entry to a tissue chamber 364, with the straight portions 360 extended distally relative to the curved portions 358. As compared to a design with a fully curved (e.g., circular or oval) opening, the straight portions 360, may allow the size of the tissue chamber to be maximized without increasing the overall diameter of the suture apparatus. The distal engagement surface 356 may be angled or skewed relative to the central axis A1. For example, the distal engagement surface may form an angle between approximately 20 and 60 degrees relative to the axis A1. This skewed angle may allow the suture apparatus 350 to approach anatomic tissue from a direction non-perpendicular to the tissue which may be a more desirable approach, requiring less severe contortion of the flexible device. The angled distal opening of the suture apparatus may also allow for a larger field of view, greater space for tissue ingress, and a gentler entry into an anatomic orifice. In some examples, the distance around the distal engagement surface 356 may be approximately the same distance as around the distal engagement surface 240 of FIG. 3. In other words, the distance around the distal engagement surface 356 may be the same as the distance around a circular distal engagement surface having a 16 mm diameter. Thus, the apparatus 350 may navigate anatomic passages that may conform the shape of the housing 354.

As shown in FIG. 7A, tissue engagement features 370 may extend from the distal engagement surface 356 or from another area near a distal portion of the suture apparatus 350. Tissue engagement features may be included in any of the suture apparatuses described herein. The tissue engagement features 370 may include, for example, prongs, spikes, tines, or other extended projections that may grip adjacent tissue. The tissue engagement features 370 may provide a counter tension to a force applied by a rotating needle 374 (which may be similar, for example, to any of needles 238, 300, 504). In some examples, the tissue engagement features 370 may be retractable so they do not snag tissue as the flexible device is navigated through anatomic passageways to the needle tissue engagement location. As shown in FIG. 7B, the tissue engagement features 370 may engage one or more layers of tissue 380. In this example, tissue 380 may include a mucosal layer 381, a submucosal layer 382, and a muscle layer 383. For example, the tissue engagement features 370 may engage the mucosal layer 381 of the tissue 380 and not engage the submucosal 382 and muscle 383 layers of the tissue. The tissue engagement features 370 may prevent sliding of the mucosal layer 381 with respect to the distal engagement surface 356, allowing for an even capture of the tissue 380 by holding the mucosal layer 381 fixed with respect to the distal engagement surface 356. As a portion of the mucosal layer 381 between the engagement features 370 is pulled by suction force into the tissue chamber 364 of the housing 354, the submucosal 382 and muscle 383 layers that are not held by the tissue engagement features 370 may slip toward or into the tissue chamber 364 under force of the vacuum. As shown in FIG. 7B, the tissue engagement features 370 may hold the tissue 380 as the needle 374 rotates through the tissue chamber 364 and through the fold of the tissue 380.

FIG. 7C illustrates a suture apparatus 400 according to an alternative example. In this example, the suture apparatus 400 may be substantially similar to the suture apparatus 204 or 350, with the differences as described. In this example, the suture apparatus 400 includes a housing 402 including a distal engagement surface 404 that may contact a tissue during a suturing procedure. In this example the distal engagement surface 404 may have an irregular shape including curved portions and straight portions. The distal engagement surface 404 may be angled or skewed relative to the central axis A1. In this example, tissue engagement features 406 may be spaced around approximately half or greater than a half of the distal engagement surface 404 to provide secure attachment to tissue. The secure attachment may provide greater counter resistance to the force of the needle pushing through the folded tissue and may prevent sliding of the mucosal layer of tissue at the distal engagement surface 404.

FIG. 7D illustrates a suture apparatus 450 according to an alternative example. In this example, the suture apparatus 450 may be substantially similar to the suture apparatus 204, 350, or 400, with the differences as described. In this example, the suture apparatus 450 includes a housing 452 including a distal engagement surface 454 that may contact a tissue during a suturing procedure. In this example the distal engagement surface 454 may be angled or skewed relative to the central axis A1 such that a length L1 of the housing is shorter than a length L2 of the housing. A viewing port 456 may extend through a shorter portion 455 of the housing 452, allowing for a larger field of view 458 for an imaging system capturing image data through viewing port 456, as compared to a viewing port 456 that would extend the full length L2. In this example, the portion 455 of the housing 452 may be constructed of a transparent material.

FIGS. 7E-7G illustrate a suture apparatus 460 according to an alternative example. In this example, the suture apparatus 460 may be substantially similar to the suture apparatus 204, 350, 400, or 450, with the differences as described. In this example, the suture apparatus 460 includes a port 462 with a tissue apposition tool 464 extending therethrough to draw tissue into a tissue chamber 465 and into a path of a needle 466. The tissue apposition tool 464 may be part of a tissue biasing component configured to draw tissue into the tissue chamber. The tissue apposition tool 464 may optionally be used in conjunction with vacuum apposition as described herein or may be used without vacuum apposition. The tissue apposition tool 464 is an example of tissue apposition tool 111. The depicted tissue apposition tool 464 includes a helical shaped component to engage tissue, while other tissue engaging arrangements may include opposed jaws, clamps, etc. FIG. 7E shows the tissue apposition tool 464 in an extended configuration distal of a housing 468 of the suture apparatus 460 to engage target tissue. FIG. 7F shows the tissue apposition tool 464 in a retracted configuration within the housing 468. FIG. 7G shows the tissue apposition tool 464 in the retracted configuration within the housing 468 and engaged with tissue such that the tissue is in position for a full thickness bite of the tissue by the needle 466. In use, suture apparatus 460 may be coupled to a distal end of an elongate flexible device (e.g., an endoscope). The suture apparatus 460 may be permanently or releasably attached to the elongate flexible device. Once coupled, the tissue apposition tool 464 may be extended distally through a working channel of the elongate flexible device, through port 462 in the suture apparatus 460, and distally past the housing 468 of the suture device 460. The tissue apposition tool 464 may be pushed forward against the tissue to engage the tissue. In embodiments where the tissue apposition tool 464 includes a helical shaped tissue engaging component, the tissue apposition tool 464 can be advanced distally while being rotated such that the helical shaped component drives into the tissue in a corkscrew movement. Once the helical shaped component has been driven into the tissue to a desired depth, the helical shaped component can be pulled back without twisting, bringing the tissue into the tissue chamber 465 and into the suturing needle path. In embodiments where the tissue apposition tool 464 includes opposing jaws, the tissue apposition tool can be advanced distally and the jaws opened to bite onto tissue. The jaws can then be closed to grab the tissue and the tissue apposition tool 464 retracted proximally to draw tissue into the tissue chamber 465. Following a suturing operation by the needle 466, the tissue apposition tool 464 can be disengaged from the tissue to release the tissue from the tissue chamber 465.

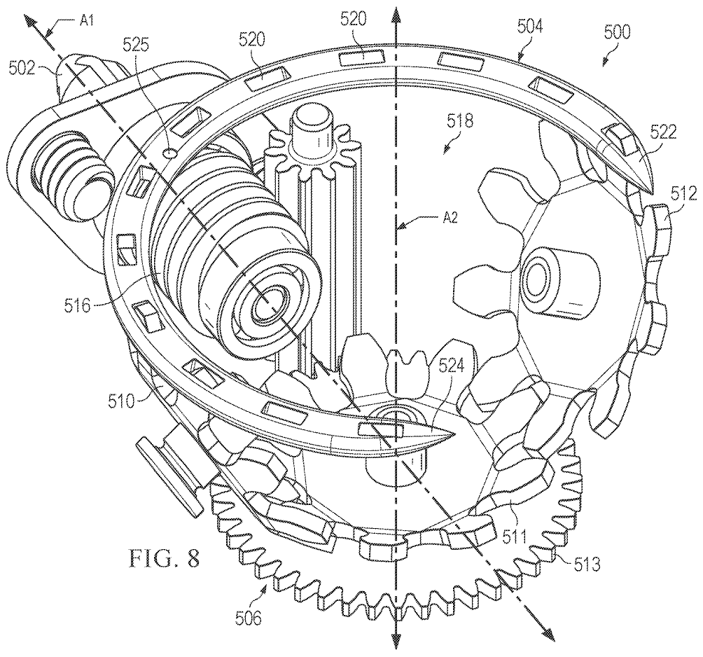

FIG. 8 illustrates a needle drive system 500 according to an alternative embodiment. In some examples the needle drive system 500 may be used in place of the needle drive system 232. In this example, the needle drive system 500 may include a drive input 502 and a gear system 506 for driving a needle 504. The gear system 506 may be coupled between the drive input 502 and the needle 504. The gear system 506 may transmit torque from the drive input 502 to drive motion of the needle 504. In this example, the gear system 506 may include three bevel gears 510, 511, 512 arranged in a generally trapezoidal configuration. Gears 510 and 512 may be drive gears that releasably engage the needle 504 to drive motion of the needle 504. The center bevel gear 511 may be fixed to a gear 513. The gear 513 may engage a proximal spur gear 514. The spur gear 514 may be engaged and driven by a worm gear 516 which may be coupled to the drive input 502. In this gear configuration, torque may be applied to the center bevel gear 511 which is generally positioned opposite the needle 504 so that torque and backlash are balanced between the two drive gears 510, 512. The trapezoidal configuration of the three bevel gears 510, 511, 512 form an open space 518 into which tissue may be folded when a vacuum is applied. The gears 511, 513 may rotate about the axis A2 that may be generally perpendicular to the axis A1. The needle 504 may include drive engagement features 520 patterned along a centerline of the needle to eliminate interaction with the engagement features and the apposed tissue, therefore reducing tissue trauma and needle drive forces. The drive engagement features 520 of the needle 504 may engage the drive gears 510, 512. The drive engagement features 520 may include slots extending through top and bottom surfaces of the needle between inner and outer circumferences of the needle 504. The needle 504 may have an arc or partially circular shape and may rotate about the axis A2 to sweep through the tissue chamber and through the tissue captured within the chamber. In some examples, the needle 504 may extend approximately 270 degrees. The needle 504 may include a pointed end 522 and a pointed end 524 to allow the needle to be driven in either a clockwise or counter-clockwise direction. The direction of rotation of the drive input 502 may drive either the clockwise or counter-clockwise motion of the needle 504. In some examples, the needle 504 may rotate in a full 360 degree circular route to return a suture-affixed portion 525 of the needle 504 to a staging configuration. In some examples, the needle 504 may pivot bi-directionally in a semi-circular route to return the suture-affixed portion 525 of the needle to the staging configuration. In the staging configuration, the needle 504 may be rotated to a position such that the needle does not obstruct ingress of tissue to the chamber. Suture material may be fixed to the needle, for example, at the suture-affixed portion 525. As the needle 504 is rotated about the axis A2, the needle 504 may draw the suture material through the folded tissue held within the tissue chamber to create a stitch. In some embodiments, the needle 238 may have a single pointed end (e.g., pointed end 522) and an opposite blunted end (e.g., end 524).

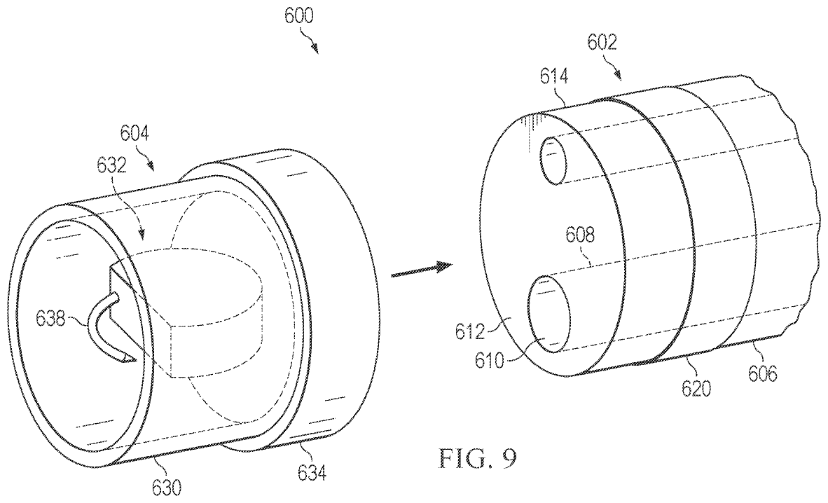

FIG. 9 illustrates a side view of a distal portion of an instrument system 600 including an elongate flexible device 602 and a suture apparatus 604. The instrument system 600 may be similar to the instrument system 100, with the differences as described. In particular, a concentric drive member may allow for a larger working channel. The device 602 may serve as a platform to drive motion of mechanisms in the suturing apparatus, introduce working components into the anatomic structures accessed by the instrument system 600, and capture image data of the anatomic structures. The device 602 may include a flexible body 606 through which extends one or more working channels 608. The working channels 608 may extend through the flexible body 606 to provide passage for removable instrument systems and tissue apposition tools, and allow instruments to be exchanged during a procedure. The working channels 608 may also or alternatively allow fluid passage or otherwise provide access between proximal and distal portions of the elongate flexible device 602. The working channels 608 may define openings 610 in a distal end portion 612 of the device 602. An imaging system 614 may also extend through the flexible body 106.

In this example a drive element 620 may be external to and concentric with the flexible body 606. The drive element 620 may couple to a motor or other drive system, such a motor of a robot-assisted manipulator. The drive element 620 may couple to a drive input 634 of the suture apparatus 604. For example, the drive input 634 may include an interior engagement surface that interfaces with an exterior engagement surface of the drive element 620. The suture apparatus 604 may also include a housing 630 that houses a needle drive system 632 and a needle 638. The drive input 634 may be housed within the housing 630 or may be coupled to an exterior of the housing 630. A rotation of the drive element 620 may provide a torque to the drive input 634 that causes the needle 638 to rotate and engage tissue folded within the suture apparatus 604. The rotation of the drive element 620 may be clockwise or counter-clockwise to provide uni-directional or bi-directional motion to the needle 638.

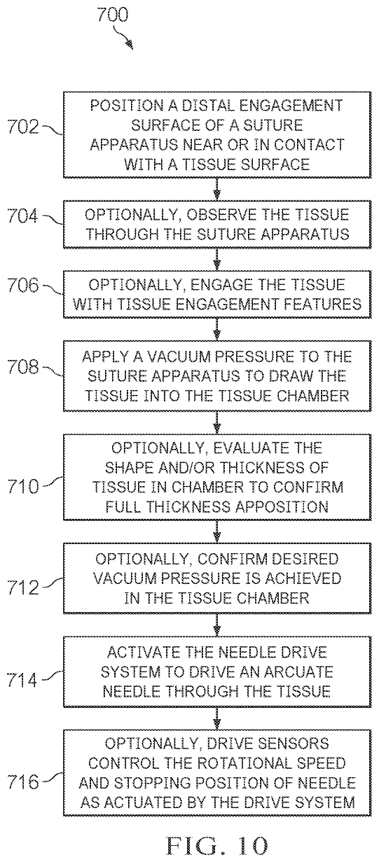

FIG. 10 is a flowchart illustrating a method 700 for suturing tissue during a medical procedure. The method 700 is illustrated as a set of operations or processes that may be performed in the same or in a different order than the order shown in FIG. 10. One or more of the illustrated processes may be omitted in some examples of the method. Additionally, one or more processes that are not expressly illustrated in FIG. 10 may be included before, after, in between, or as part of the illustrated processes. In some examples, one or more of the processes of method 700 may be implemented, at least in part, by a control system executing code stored on non-transitory, tangible, machine-readable media that when run by one or more processors (e.g., the processors of a control system) may cause the one or more processors to perform one or more of the processes. The method 700 may be used in conjunction with any of the suture apparatuses disclosed herein.

At a process 702, a distal engagement surface of a suture apparatus may be positioned near or in contact with a tissue surface. For example, an instrument system 100 may be used to perform the suture procedure. The flexible device 102 with the permanent or removably couplable suture apparatus 104. may be inserted through a patient's mouth, into the esophagus, and into the stomach. The distal end of the flexible device 102 and the suture apparatus 104 may be steered to orient the distal engagement surface 140 into contact with or in close proximity to the mucosal layer of the tissue.

At an optional process 704, the tissue distal of the suture apparatus may be viewed in the field of view of an imaging system. For example, the imaging system 118 may view the tissue through the viewing port 146 or through the suture housing 130 if a portion of the housing is constructed of a transparent material or includes a transparent window. Images of the tissue may allow a clinician to determine if the device should be repositioned or reoriented, to observe any obstructions to the suture, to observe any tissue lesions, or to observe any other circumstances that may impact the suturing procedure.

At an optional process 706, tissue engagement features extending from the suture apparatus may engage the tissue. For example, the tissue engagement features 370, 406 may extend into the tissue to resist or prevent movement of the tissue relative to the suture apparatus when the needle is driven through the full tissue thickness. In some examples the tissue engagement features may engage less than all of the layers of the tissue. For example, the tissue engagement features may engage the mucosal layer but not the submucosal or muscle layers, allowing the unengaged layers to be suctioned into the tissue chamber as the engaged layer remains fixed relative to the suture apparatus. The process 706 may optionally include extending and retracting deployable tissue engagement features.

At a process 708, a vacuum pressure may be applied to draw the tissue into the tissue chamber of the suture apparatus. For example, a vacuum pressure may be applied through the tissue biasing component 144 to create suction that draws the tissue into the chamber 142. The vacuum pressure and the shape of the tissue chamber may cause tissue apposition by forming the tissue into a convex fold within the tissue chamber. The tissue chamber may be within the field of view of the imaging system allowing the clinician to observe the sufficiency of the tissue ingress prior to initiating needle engagement with the apposed tissue. Optionally, a tissue apposition tool having a tissuing engaging component may be extended to grab onto target tissue and retracted to draw tissue into the tissue chamber 142. The tissue apposition tool may be used in addition to or instead of vacuum pressure to draw the tissue into the tissue chamber.

At an optional process 710, the shape and/or thickness of the tissue in the chamber may be evaluated to confirm full thickness apposition. For example, the sensor system 257 may include an imaging sensor such as an ultrasound sensor, positioned on the bottom surface of the tissue chamber and pointed in the direction of A2, which may be used to evaluate the shape or thickness of tissue in the tissue chamber to verify that a full thickness of tissue has been captured prior to suture.

At an optional process 712, a confirmation may be made that a desired vacuum pressure has been achieved in the tissue chamber. For example, the sensor system 257 may include a pressure sensor that may be used to confirm an expected vacuum pressure.

At a process 714, the needle drive system may be activated to drive the needle through the apposed tissue. For example, with the tissue in the chamber 142, the drive element 124 may be actuated to activate the needle drive system 132 to move the arcuate needle 138 and the attached suture material through the tissue. More specifically, the drive element 124 may provide torque to the drive input 134 which causes the gear system 136 to operate to rotate the needle 138 in a uni-directional or bi-directional path to draw suture material through the apposed tissue in the tissue chamber 142. The processes 702-710 may be repeated as needed to accomplish the surgical goal.

At an optional process 716, drive sensors may be utilized to control the rotational speed and stopping position of the needle as actuated by the drive system. For example, the sensor system 257 may include one or more drive sensors, such as a plurality of hall effect sensors, inductive proximity sensors, and/or optical sensors functioning as an encoder to measure rotations of the needle either directly or by inference. Sensed measurements may occur at any moving element of the drive system.

The suturing procedure may achieve a full thickness suture throw through apposed tissue including all layers of the tissue. For example, in a stomach suturing procedure, the needle and suture may penetrate all three layers of the tissue including the submucosal, mucosal, and muscle layers.

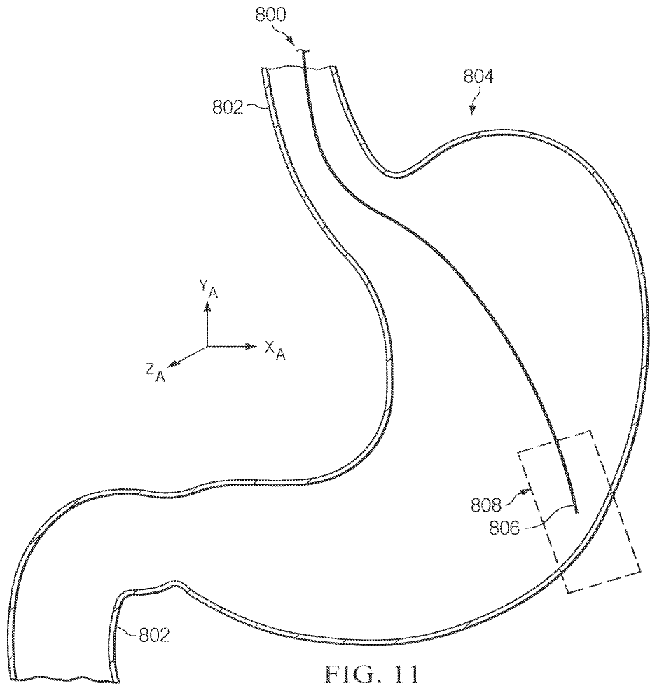

FIG. 11 illustrates an endoscopic instrument system 800 (e.g., the instrument system 100) extending within anatomic passageways 802 of an anatomical structure 804. In some examples the anatomic structure 804 may be a stomach. The anatomic structure 804 has an anatomical frame of reference (XA, YA, ZA). A distal end portion 806 of the endoscopic instrument system 800 may be advanced into an anatomic opening (e.g., a patient mouth) and through the anatomic passageways 802 to perform a medical procedure, such as a suturing procedure, at or near target tissue located in a region 808 of the anatomic structure 804 using any of the methods or systems described herein.

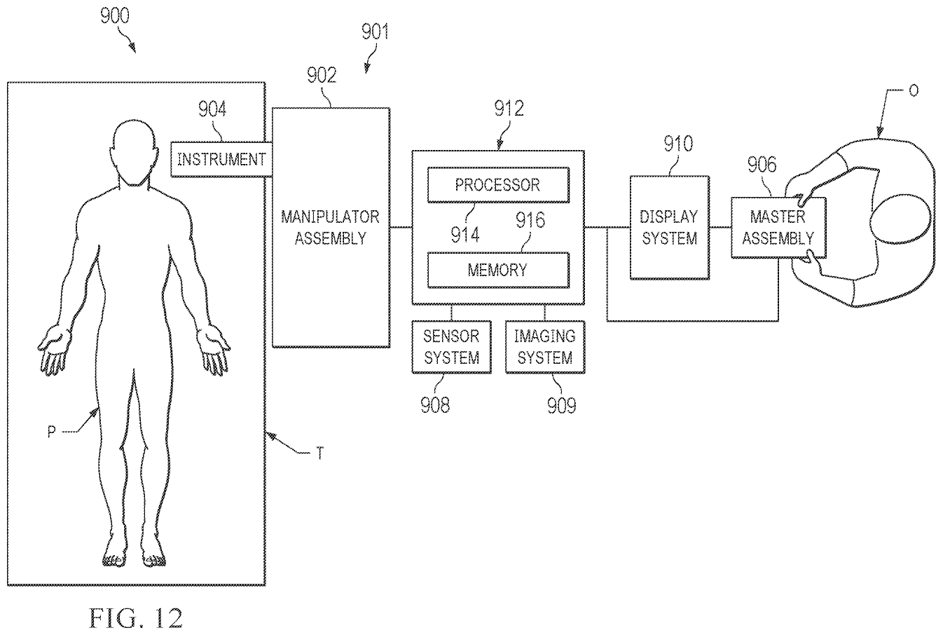

FIG. 12 illustrates a robotically-assisted medical system, according to some examples. In some examples, the systems and methods disclosed herein may be used in a suturing procedure performed with a robotically-assisted medical system as described in further detail below. As shown in FIG. 12, a robotically-assisted medical system 900 may include a manipulator assembly 902 for operating a medical instrument 904 (e.g., instrument system 100, 200 or any of the instruments described herein) in performing various procedures on a patient P positioned on a table T in a surgical environment 901. For example, the manipulator assembly 902 may engage the drive element 224 to provide torque to the drive input 234 of the suture apparatus 204. The manipulator assembly 902 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. A master assembly 906, which may be inside or outside of the surgical environment 901, generally includes one or more control devices for controlling manipulator assembly 902. Manipulator assembly 902 supports medical instrument 904 and may optionally include a plurality of actuators or motors that drive inputs on medical instrument 904 in response to commands from a control system 912. The actuators may optionally include drive systems that when coupled to medical instrument 904 may advance medical instrument 904 into a naturally or surgically created anatomic orifice. Other drive systems may move the distal end of medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). The manipulator assembly 902 may support various other systems for irrigation, treatment, or other purposes. Such systems may include fluid systems (including, for example, reservoirs, heating/cooling elements, pumps, and valves), generators, lasers, interrogators, and ablation components.

Robotically-assisted medical system 900 also includes a display system 910 for displaying an image or representation of the surgical site and medical instrument 904 generated by an imaging system 909 which may include an endoscopic imaging system. Display system 910 and master assembly 906 may be oriented so an operator O can control medical instrument 904 and master assembly 906 with the perception of telepresence. Any of the previously described graphical user interfaces may be displayable on a display system 910 and/or a display system of an independent planning workstation.

In some examples, the endoscopic imaging system components of the imaging system 909 may be integrally or removably coupled to medical instrument system 904. However, in some examples, a separate endoscope, attached to a separate manipulator assembly may be used with medical instrument system 904 to image the surgical site. The endoscopic imaging system 909 may be implemented as hardware, firmware, software, or a combination thereof which interact with or are otherwise executed by one or more computer processors, which may include the processors of the control system 912.

The sensor system 908 (which may include the sensor system 257) may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument 904. The sensor system 908 may also include temperature, pressure, force, or contact sensors or the like.

Robotically-assisted medical system 900 may also include control system 912. Control system 912 includes at least one memory 916 and at least one computer processor 914 for effecting control between medical instrument 904, master assembly 906, sensor system 908, and display system 910. Control system 912 also includes programmed instructions (e.g., a non-transitory machine-readable medium storing the instructions) to implement a suturing procedure using the robotically-assisted medical system including for navigation, steering, imaging, engagement feature deployment or retraction, and driving the needle.

Control system 912 may optionally further include a virtual visualization system to provide navigation assistance to operator O when controlling medical instrument 904 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 912 may use a pre-operative image to locate the target tissue (using vision imaging techniques and/or by receiving user input) and create a pre-operative plan, including an optimal first location for performing bronchial passageway and vasculature occlusion. The pre-operative plan may include, for example, a planned size to expand the expandable device, a treatment duration, a treatment temperature, and/or multiple deployment locations.

In the description, specific details have been set forth describing some examples. Numerous specific details are set forth to provide a thorough understanding of the examples. It will be apparent, however, to one skilled in the art that some examples may be practiced without some or all these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions. Not all the illustrated processes may be performed in all examples of the disclosed methods. Additionally, one or more processes that are not expressly illustrated in may be included before, after, in between, or as part of the illustrated processes. In some examples, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging and treatment, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

One or more elements in examples of this disclosure may be implemented in software to execute on a processor of a computer system such as control processing system. When implemented in software, the elements of the examples of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

Note that the processes and displays presented might not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

This disclosure describes various instruments, portions of instruments, and anatomic structures in terms of their state in three-dimensional space. As used herein, the term position refers to the location of an object or a portion of an object in a three-dimensional space (e.g., three degrees of translational freedom along Cartesian x-, y-, and z-coordinates). As used herein, the term orientation refers to the rotational placement of an object or a portion of an object (e.g., in one or more degrees of rotational freedom such as roll, pitch, and/or yaw). As used herein, the term pose refers to the position of an object or a portion of an object in at least one degree of translational freedom and to the orientation of that object or portion of the object in at least one degree of rotational freedom (e.g., up to six total degrees of freedom). As used herein, the term shape refers to a set of poses, positions, or orientations measured along an object.

While certain illustrative examples of the invention have been described and shown in the accompanying drawings, it is to be understood that such examples are merely illustrative of and not restrictive on the broad invention, and that the examples of the invention are not limited to the specific constructions and arrangements shown and described, since various other modifications may occur to those ordinarily skilled in the art.

1. An apparatus comprising:
a housing including an instrument interface, the instrument interface configured to releasably engage a distal portion of an endoscope;
a drive system coupled to the housing and configured to releasably engage a drive element extending from the endoscope;
an arc-shaped needle coupled to the drive system, wherein the drive element is configured to provide motion to the arc-shaped needle when the drive system is engaged with the drive element; and
a tissue apposition system comprising:
a tissue chamber in the housing across which a trajectory of the arc-shaped needle extends; and
a tissue biasing component configured to draw tissue into the tissue chamber.

2. The apparatus of claim 1, wherein the drive system is configured to provide bi-directional motion to the arc-shaped needle.

3. The apparatus of claim 1, wherein the housing includes a generally transparent material through which a field of view of the endoscope is visible.

4. The apparatus of claim 1, wherein a proximal end of the housing includes an attachment feature configured to interface with the endoscope.

5. (canceled)

6. The apparatus of claim 1, wherein the drive system includes a drive input at a proximal end of the housing, the drive input configured to releasably engage the drive element extending from the endoscope.

7. The apparatus of claim 6, wherein the drive element comprises a distal drive head and a drive shaft, the drive element configured to be inserted into a working channel of the endoscope for releasably coupling the distal drive head to the drive input at the proximal end of the housing.

8. The apparatus of claim 6, wherein the drive system includes a gear system that interfaces between the drive input and the arc-shaped needle.

9. The apparatus of claim 8, wherein the gear system includes a central gear and at least two drive gears on opposite sides of the tissue chamber.

10-11. (canceled)

12. The apparatus of claim 1, wherein the tissue chamber is sized to receive a full thickness fold of a tissue.

13. The apparatus of claim 1, wherein the arc-shaped needle has a needle trajectory that extends 360 degrees in a plane approximately parallel to a longitudinal axis of the housing.

14. The apparatus of claim 1, wherein the arc-shaped needle has a needle trajectory that approximately bisects the tissue chamber.

15. The apparatus of claim 1, wherein the tissue biasing component includes an aperture through which a vacuum force is applied to draw the tissue into the tissue chamber.

16. The apparatus of claim 1, wherein the tissue biasing component comprises a tissue apposition tool configured to be advanced to bite onto tissue and retracted to draw the tissue into the tissue chamber.

17. The apparatus of claim 16, wherein the tissue apposition tool comprises a helical shaped component to engage tissue.

18. (canceled)

19. The apparatus of claim 1, wherein the housing includes a viewing port configured to align with an imaging system of the endoscope.

20. (canceled)

21. The apparatus of claim 1, wherein the arc-shaped needle extends approximately 270 degrees.

22. The apparatus of claim 1, wherein the arc-shaped needle comprises a first pointed end and a second pointed end; wherein when the drive system is rotated in a first direction, the first pointed end is a leading end of the arc-shaped needle; and when the drive system is rotated in a second direction opposite to the first direction, the second pointed end is a leading end of the arc-shaped needle.

23. The apparatus of claim 1, wherein the arc-shaped needle includes an attachment portion for attachment to a suture.

24. The apparatus of claim 1, wherein the arc-shaped needle includes teeth along an inner diameter of the needle configured to engage the drive system or the arc-shaped needle includes a slotted rack configured to engage the drive system.

25. (canceled)

26. The apparatus of claim 1, wherein the arc-shaped needle includes a releasable suture tag.

27. The apparatus of claim 1, wherein the housing includes a track in which the arc-shaped needle is supported.

28. The apparatus of claim 1, wherein the arc-shaped needle has a trajectory that remains entirely within the housing.

29. The apparatus of claim 1, wherein the arc-shaped needle has a trajectory that extends beyond a distal end of the housing.

30-36. (canceled)

37. The apparatus of claim 1, further comprising a suture tag mechanism including a suture cartridge and a suture release mechanism.

38-61. (canceled)
